(19) United States
(12) Patent Application Publication
(10) Pub. No.: US 2026/0011410 A1
(43) Pub. Date: Jan. 8, 2026

(54) METHOD AND SYSTEM FOR PREDICTING COMMUNITY DYNAMIC CHANGE OF LONG-PERIOD ENRICHED BACTERIAL COMMUNITY IN COLD SEEP

(71) Applicants: GUANGDONG UNIVERSITY OF TECHNOLOGY, Guangdong (CN); GUANGDONG LABORATORY OF SOUTHERN OCEAN SCIENCE AND ENGINEERING (GUANGZHOU), Guangdong (CN)

(72) Inventors: Jingchun FENG, Guangdong (CN); Si ZHANG, Guangdong (CN); Yuhang ZHANG, Guangdong (CN); Yan XIE, Guangdong (CN)

(73) Assignees: GUANGDONG UNIVERSITY OF TECHNOLOGY, Guangdong (CN); GUANGDONG LABORATORY OF SOUTHERN OCEAN SCIENCE AND ENGINEERING (GUANGZHOU), Guangdong (CN)

(21) Appl. No.: 19/260,314

(22) Filed: Jul. 4, 2025

(30) Foreign Application Priority Data
Jul. 5, 2024 (CN) 202410897030.0

(51) Int. Cl. G16B 40/10; G16B 25/20

(52) U.S. Cl. CPC G16B 40/10; G16B 25/20

(57) ABSTRACT

A method and system for predicting a community dynamic change of a long-period enriched bacterial community in a cold seep. The method includes: collecting Raman spectra of a bacterial culture sample at different enrichment stages, analyzing them using an MCR-ALS algorithm to acquire metabolite data, and storing the metabolite data, environmental parameters, bacterial community α-diversity, and relative abundances of key bacterial taxa jointly as an original dataset; performing importance calculation and data screening on the original dataset using a CART decision tree algorithm; training a random forest prediction model for prediction using the screened dataset; and finally performing community dynamic change prediction using a trained random forest model. According to the present disclosure, a dataset is formed through routine sampling and collection of a complete bacterial community succession process in an early stage of enrichment culture, and an accurate prediction model is constructed by using a machine learning algorithm.

### CROSS-REFERENCE TO RELATED APPLICATION

This application claims the priority benefit of China application serial no. 202410897030.0, filed on Jul. 5, 2024. The entirety of the above-mentioned patent application is hereby incorporated by reference herein and made a part of this specification.

### BACKGROUND

### Technical Field

The present disclosure relates to the technical field of microbial monitoring, and more particularly, to a method and system for predicting a community dynamic change of a long-period enriched bacterial community in a cold seep.

### Description of Related Art

Cold seep ecosystems harbor abundant functional bacterial taxa such as methane-oxidizing bacteria, sulfate-reducing bacteria, and methanogens, which rely on cold seep seepage for survival and reproduction. Their unique biochemical processes and metabolites significantly mitigate the greenhouse effect caused by methane migration into the atmosphere, while also providing important scientific evidence for exploring biogeochemical cycles in extreme environments and even the origin of life.

However, cold seep environments exhibit deep-sea extreme characteristics such as high pressure, low oxygen, and high-concentration methane seepage, making it difficult for cold seep bacteria to normally grow and reproduce in terrestrial environments. Existing studies have achieved successful enrichment culture of key cold seep bacterial taxa through integrity-preserving sampling of cold seep samples and laboratory simulation of high-pressure and low-temperature conditions. During the enrichment culture period, regular monitoring of dynamic changes in metabolites and bacterial communities in the enrichment system is an important indicator for indicating the successful enrichment of key bacterial taxa. However, the reproduction and iteration cycle of cold seep bacteria is generally longer than that of bacteria in normal environments. Studies have reported that the enrichment cycle of cold seep bacteria may take months or even years.

The prior art typically adopts regular sampling, DNA extraction, and library construction and sequencing to monitor enriched bacterial communities. Nevertheless, the DNA extraction requires a high quantity demand for enriched samples. The ultra-long enrichment culture cycle (such as several years) limits the sampling volume and frequency of the enrichment system. Meanwhile, the collected enriched samples may fail to capture the “inflection points” of key bacterial growth, metabolism, and community dynamic changes in the enriched samples due to bacteria not reaching the reproduction cycle, resulting in inefficient sampling and greatly affecting the sampling and monitoring efficiency of enriched samples.

### SUMMARY

To overcome the problems of a large sampling volume, a low frequency, and low efficiency in the traditional monitoring of community dynamics of enriched bacterial communities in cold seeps in the above prior art, the present disclosure provides a method and system for predicting a community dynamic change of a long-period enriched bacterial community in a cold seep. A dataset is formed through routine sampling and collection of a complete bacterial community succession process (adaptation, growth, reproduction, and stabilization) in an early stage of enrichment culture, and an accurate prediction model is constructed by using a machine learning algorithm, thereby improving sampling efficiency of a long-period enrichment culture operation for a cold seep bacterial community.

To solve the above technical problems, the technical solutions of the present disclosure are as follows:

A method for predicting a community dynamic change of a long-period enriched bacterial community in a cold seep, including steps of:
S1: collecting Raman spectra, environmental parameters, bacterial community α-diversity, and relative abundances of key bacterial taxa of a bacterial culture sample at different enrichment stages;
analyzing the Raman spectra using a multivariate curve resolution-alternating least squares (MCR-ALS) algorithm to acquire metabolite data; and
storing the metabolite data, the environmental parameters, the bacterial community α-diversity, and the relative abundances of key bacterial taxa jointly as an original dataset;
S2: performing importance calculation and data screening on the original dataset using a CART decision tree algorithm (i.e., Classification and Regression Trees algorithm) to acquire a screened dataset;
S3: optimizing and training a random forest prediction model for prediction using the screened dataset to acquire a trained random forest prediction model; and
S4: acquiring metabolite data and environmental parameters of a bacterial culture sample to be predicted, inputting the metabolite data and environmental parameters of a bacterial culture sample to be predicted jointly into the trained random forest prediction model, and outputting, by the trained random forest prediction model, prediction results of bacterial community α-diversity and relative abundances of key bacterial taxa.

Preferably, in the step S1, enrichment stages of bacteria include: an adaptation stage, a growth stage, a reproduction stage, and a stabilization stage.

Preferably, in the step S1, the environmental parameters include: water quality physicochemical parameters, a nutrient salt content, and OD600 bacterial concentration parameters;
the bacterial community α-diversity includes: a Shannon index, a Chao1 index, an Ace index, and a Simpson index;
the relative abundances of key bacterial taxa include: relative abundances of Anaerobic Methanotrophic Archaea (ANME), sulfate-reducing bacteria (SRB), and methanogens; and
the metabolite data include: contents of formic acid, acetic acid, methanol, formaldehyde, and sulfate.

Preferably, the water quality physicochemical parameters include: contents of dissolved methane and carbon dioxide, temperature, salinity, pH, and a dissolved oxygen content; and
the nutrient salt content includes: contents of nitrate, dissolved organic matter (DOM), and total organic carbon (TOC).

Preferably, the Raman spectra are collected using a confocal Raman microspectroscopic probe;
the water quality physicochemical parameters are collected using a multi-parameter water quality probe;
the nutrient salt content and the OD600 bacterial concentration parameters are collected using an ultraviolet spectrophotometric probe; and
DNA extraction, PCR amplification, library construction, and sequencing are sequentially performed on the bacterial culture sample at different enrichment stages to acquire DNA sequencing data; and the bacterial community α-diversity and the relative abundances of key bacterial taxa are acquired according to the DNA sequencing data.

Preferably, in the step S1, the method further includes: evaluating analysis accuracy of the MCR-ALS algorithm using an MCR-BANDS algorithm.

Preferably, the step S2 includes:
constructing a decision tree using the original dataset, and calculating a Gini coefficient of each decision node in the decision tree using the CART decision tree algorithm;
calculating a variable importance measure (VIM) of each data in the original dataset according to the Gini coefficient; and
performing the data screening according to the VIM, and eliminating data with a VIM lower than a preset threshold to acquire the screened dataset.

Preferably, the step S3 includes:
dividing the screened dataset into a training set and a testing set;
training the random forest prediction model using the training set to acquire a random forest prediction model after training; and
evaluating performance parameters of the random forest prediction model after training using the testing set, and optimizing the random forest prediction model after training iteratively until the performance parameters meet preset conditions to acquire the trained random forest prediction model.

Preferably, the screened dataset is divided into the training set and the testing set at a ratio of 9:1 using a ten-fold cross-validation method;
the performance parameters include: a coefficient of determination, a root mean square error, and a mean absolute error; and
when the coefficient of determination is larger, and the root mean square error and the mean absolute error are smaller, then performance of the random forest prediction model after training is better.

The present disclosure further provides a system for predicting a community dynamic change of a long-period enriched bacterial community in a cold seep, applying the method for predicting a community dynamic change of a long-period enriched bacterial community in a cold seep described above, and including:
a data acquisition unit: configured to collect Raman spectra, environmental parameters, bacterial community α-diversity, and relative abundances of key bacterial taxa of a bacterial culture sample at different enrichment stages;
analyze the Raman spectra using an MCR-ALS algorithm to acquire metabolite data; and
store the metabolite data, the environmental parameters, the bacterial community a-diversity, and the relative abundances of key bacterial taxa jointly as an original dataset;
a data screening unit: configured to perform importance calculation and data screening on the original dataset using a CART decision tree algorithm to acquire a screened dataset;
a model training unit: configured to optimize and train a random forest prediction model for prediction using the screened dataset to acquire a trained random forest prediction model; and
a community dynamic change prediction unit: configured to acquire metabolite data and environmental parameters of a bacterial culture sample to be predicted, input the metabolite data and environmental parameters of the bacterial culture sample jointly into the trained random forest prediction model, and output, by the trained random forest prediction model, prediction results of bacterial community α-diversity and relative abundances of key bacterial taxa.

Compared with the prior art, the beneficial effects of the technical solutions of the present disclosure are as follows:

The present disclosure provides a method and system for predicting a community dynamic change of a long-period enriched bacterial community in a cold seep. First, Raman spectra, environmental parameters, bacterial community α-diversity, and relative abundances of key bacterial taxa in a long-period bacterial culture sample at different enrichment stages are collected. The Raman spectra are analyzed using an MCR-ALS algorithm to acquire metabolite data. The metabolite data, the environmental parameters, the bacterial community α-diversity, and the relative abundances of key bacterial taxa are stored jointly as an original dataset. Next, importance calculation and data screening are performed on the original dataset using a CART decision tree algorithm to acquire a screened dataset. Then a random forest prediction model is optimized and trained using the screened dataset to acquire a trained random forest prediction model. Finally, metabolite data and environmental parameters of a bacterial culture sample to be predicted are acquired and input jointly into the trained random forest prediction model, and the trained random forest prediction model outputs prediction results of bacterial community a-diversity and relative abundances of key bacterial taxa.

The present disclosure has the following advantages:
1) According to the present disclosure, qualitative and quantitative analysis of compound components and concentrations thereof in a culture sample is realized through Raman spectra and an MCR-ALS algorithm. Compared with existing bacterial metabolite monitoring technologies, it avoids sample pretreatment processes such as concentration and extraction, enabling efficient, accurate, and rapid detection of characteristic metabolites in different enriched samples.
2) According to the present disclosure, a dataset is constructed using multiple parameter data from a long-period enrichment stage of a cold seep sample, and a constructed random forest prediction model is screened and trained to achieve high-precision prediction of enriched bacterial community diversity. This can indicate a dynamic change of a bacterial community and effectively identify an “inflection point” in a long-period culture stage of a cold seep.
3) According to the present disclosure, a random forest model is established to specifically predict and output abundances of key functional bacteria in an enriched bacterial community of a cold seep. Since a dataset of a prediction model is formed by integrating continuous culture monitoring of a same enrichment system, compared with a non-continuous dataset in a database, functional bacteria and environmental parameter changes of the enriched bacterial community exhibit continuity and periodicity. Adding the abundances of the key functional bacteria as a prediction output can more accurately indicate a dynamic change of the enriched bacterial community over a long period, thereby formulating a more targeted sampling strategy to improve accuracy and efficiency of a sampling operation.
4) Compared with existing deep-sea bacterial culture technologies, the method for predicting a community dynamic change of a long-period enriched bacterial community in a cold seep of the present disclosure optimizes the problem of high sample consumption in monitoring of dynamics of a bacterial community during long-period culture of deep-sea bacteria. Meanwhile, it achieves high-precision detection without requiring pretreatment processes such as DNA extraction and PCR library construction and sequencing, efficiently, accurately, and rapidly indicating a dynamic change of a bacterial community in a sample at different enrichment stages over a long period.

### BRIEF DESCRIPTION OF THE DRAWINGS

FIG. 1 is a flow chart of a method for predicting a community dynamic change of a long-period enriched bacterial community in a cold seep provided in Embodiment 1.

FIG. 2 is an architecture diagram of a method for predicting a community dynamic change of a long-period enriched bacterial community in a cold seep provided in Embodiment 2.

FIG. 3 is a structural diagram of a system for predicting a community dynamic change of a long-period enriched bacterial community in a cold seep provided in Embodiment 2.

### DESCRIPTION OF THE EMBODIMENTS

The drawings are for exemplary illustration only and shall not be construed as limitations to this patent.

To better illustrate this embodiment, certain components in the drawings may be omitted, enlarged, or reduced, and do not represent the dimensions of the actual product.

For those skilled in the art, it is understandable that certain well-known structures in the drawings and descriptions thereof may be omitted.

The technical solutions of the present disclosure are further illustrated below in conjunction with the drawings and embodiments.

### Embodiment 1

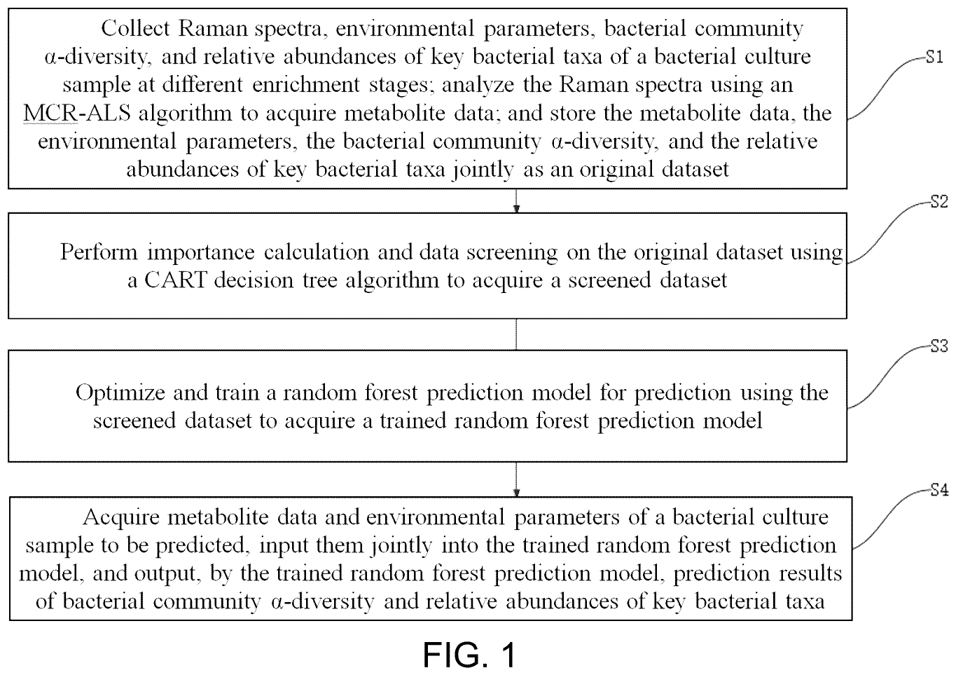

As shown in FIG. 1, this embodiment provides a method for predicting a community dynamic change of a long-period enriched bacterial community in a cold seep, including steps of:
S1: collecting Raman spectra, environmental parameters, bacterial community a-diversity, and relative abundances of key bacterial taxa of a bacterial culture sample at different enrichment stages;
analyzing the Raman spectra using a multivariate curve resolution-alternating least squares (MCR-ALS) algorithm to acquire metabolite data; and
storing the metabolite data, the environmental parameters, the bacterial community a-diversity, and the relative abundances of key bacterial taxa jointly as an original dataset;
S2: performing importance calculation and data screening on the original dataset using a CART decision tree algorithm to acquire a screened dataset;
S3: optimizing and training a random forest prediction model for prediction using the screened dataset to acquire a trained random forest prediction model; and
S4: acquiring metabolite data and environmental parameters of a bacterial culture sample to be predicted, inputting the metabolite data and environmental parameters of the bacterial culture sample to be predicted jointly into the trained random forest prediction model, and outputting, by the trained random forest prediction model, prediction results of bacterial community α-diversity and relative abundances of key bacterial taxa.

In a specific implementation process, this embodiment performs prediction on a community dynamic change of a long-period enriched bacterial community in a cold seep based on machine learning. As an intelligent prediction algorithm, machine learning has accumulated a large number of algorithms and applications in the field of microbial community prediction. It can train and construct algorithm models based on existing datasets, and then predict the dynamic change laws of subsequent stages. The simulation prediction of machine learning can avoid the problems of a large sampling volume and a low frequency caused by routine sampling and sequencing monitoring. Therefore, machine learning has good adaptability to the ultra-long cycle enriched samples in cold seeps. It can construct a model through the dataset collected by sampling in an early stage of enrichment culture, predict the change trend of the bacterial community in a subsequent enrichment stage to make a reasonable decision, and achieve efficient sampling operations.

First, Raman spectra, environmental parameters, bacterial community α-diversity, and relative abundances of key bacterial taxa of a long-period bacterial culture sample are collected at different enrichment stages.

The Raman spectra are analyzed using a multivariate curve resolution-alternating least squares (MCR-ALS) algorithm to acquire metabolite data.

An MCR-ALS algorithm model is as follows:

$$D = {CS}^{T} + E$$

where D is measurement data, i.e., a Raman spectral data matrix of a characteristic metabolite; C is a concentration matrix, i.e., concentration curves of different metabolites; S^{T} is a pure substance response matrix, i.e., a spectral response matrix of a pure characteristic metabolite; and E is a residual matrix of the model.

The MCR-ALS algorithm can decompose the unknown complex spectral data of a mixed sample into pure components and concentrations through a bilinear model, and has good adaptability to a culture sample with complex components. Therefore, it can compare the spectral differences between enriched samples to analyze the changes of bacterial metabolites. Raman spectral data collected are divided into multiple parts, and spectral signals and corresponding concentrations of various compounds in the bacterial culture sample at different enrichment stages are obtained based on resolution with the MCR-ALS algorithm. Robustness of the model is preliminarily evaluated according to an R² value (>99%) and an LOF (Local Outlier Factor) value (<10%) of the algorithm.

The MCR-ALS algorithm model is evaluated using an MCR-BANDS algorithm. A signal component contribution function fₙ of the MCR-BANDS algorithm under specific constraints is used to represent a relative contribution value of each component in a mixture, which can represent a degree of rotational blur and evaluate accuracy of the MCR-ALS algorithm model.

An MCR-BANDS algorithm model is as follows:

$${f}_{n} = \frac{‖ {c}_{n} {s}_{n}^{T} ‖}{‖ {cs}^{T} ‖}$$

where fₙ is a scalar value indicating a relative contribution value of a signal value of a certain characteristic component to an overall signal of n components acquired in a mixed sample;

$$‖ {C}_{n} {S}_{n}^{T} ‖$$

represents the signal value of the characteristic component, and ∥CS^{T}∥ represents all signals of the mixed sample.

When a signal of a certain characteristic component has a certain ambiguity, the MCR-BANDS algorithm can obtain maximum and minimum values of fₙ, i.e.,

$${f}_{n}^{max} and {f}_{n}^{min} ,$$

by calculating a relative contribution of each component. A difference ΔSCCF (a range of the difference is 0-1) is calculated based on maximum and minimum values of a relative contribution of the characteristic component, and a degree of rotational blur of each characteristic component can be evaluated to characterize accuracy. When the maximum and minimum values of fₙ are equal, that is, ΔSCCF is 0, it indicates that the characteristic component has no remaining degree of rotational blur, that is, the characteristic component obtains a unique solution through iteration of the MCR-ALS algorithm model. Based on the ΔSCCF value of the MCR-BANDS algorithm, accuracy of analysis results of each part of characteristic components of each part of Raman spectral data by the MCR-ALS algorithm model is further evaluated, so as to realize accurate qualitative and quantitative resolution of metabolite composition and concentrations of an enriched bacterial community sample.

Based on metabolite composition and concentration data of a sample at different enrichment stages (adaptation, growth, reproduction, and stabilization), metabolite difference data of each sample is formed.

The metabolite data, the environmental parameters, the bacterial community α-diversity, and the relative abundances of key bacterial taxa are stored jointly as an original dataset.

Subsequently, a random forest prediction model is established based on a random forest algorithm. An input of the prediction model is the Raman spectra, the environmental parameters, and the metabolite data of bacteria, and an output is the bacterial community α-diversity and the relative abundances of key bacterial taxa. The bacterial community α-diversity is used to indicate a dynamic change of a bacterial community.

The random forest algorithm can perform importance ranking and screening on multi-parameter data, and remove redundant parameters by iteration, thereby improving accuracy of the prediction model. This algorithm has high tolerance to outliers among multiple environmental parameters and is not prone to overfitting, showing good matching in prediction models, especially in prediction studies on bacterial communities. Aiming at periodic and continuous characteristics of a cold seep enrichment cycle in input data, this method proposes a specific model prediction output indicator (relative abundances of key functional bacteria), which can more accurately identify a key inflection point in the enrichment cycle.

Next, importance calculation and data screening are performed on the original dataset using a CART decision tree algorithm to acquire a screened dataset. Random forest constructs a model based on the CART decision tree as a basic algorithm. This algorithm divides values of a splitting attribute into 2 subsets, calculates a Gini index as a characteristic parameter based on a training dataset, and divides the training dataset using the principle of minimum Gini index.

By calculating the importance of each input parameter and iterating repeatedly to screen key parameters affecting a model output value, optimal parameter data are obtained for subsequent model training, which can effectively improve a model training effect and prediction precision after training completion.

Then the random forest prediction model is optimized and trained using the screened dataset. Model parameters (number of sub-datasets, maximum number of features, etc.) are adjusted to improve model accuracy, and a trained random forest prediction model is acquired.

Finally, during long-period enrichment of samples in the cold seep, real-time online monitoring is regularly performed on the samples to acquire metabolite data and environmental parameters of a bacterial culture sample to be predicted. Online monitoring data are subsequently input jointly into the trained random forest prediction model, and the trained random forest prediction model outputs prediction results of bacterial community α-diversity and relative abundances of key bacterial taxa, indicating a dynamic change in a bacterial community of the enriched sample to identify an inflection point of the change in the bacterial community, so as to formulate a targeted sampling strategy to improve sampling efficiency of the enriched sample in the cold seep.

In this method, decomposition is performed based on Raman spectral data of enriched samples and the MCR-ALS algorithm, enabling analysis of pure compound components in the complex enriched samples, achieving qualitative identification and quantitative resolution of characteristic metabolites, and characterizing metabolic differences between the enriched samples. This avoids sample concentration and extraction operations required in traditional metabolite detection processes, more efficiently, accurately, and rapidly forming a metabolite dataset for subsequent construction of a random forest prediction model.

In this method, a random forest prediction model is constructed based on multi-source data to achieve accurate prediction of bacterial community diversity indices and abundances of key functional bacteria, indicating a dynamic change in a bacterial community of a long-period enrichment system. This can be used to efficiently identify an “inflection point” in a long-period enrichment stage of the cold seep, so as to formulate a targeted sampling strategy to improve the sampling efficiency of the enriched sample.

In this method, a prediction model through the random forest algorithm is constructed to achieve rapid, efficient, and accurate prediction of community dynamics of the enriched sample, optimizing the high sample consumption in monitoring of dynamics of a bacterial community during long-period culture of deep-sea bacteria. This can be applied to indicate periodic succession and sampling decisions of a sample at different enrichment stages (adaptation, growth, reproduction, and stabilization) over a long period.

### Embodiment 2

This embodiment provides a method for predicting a community dynamic change of a long-period enriched bacterial community in a cold seep, including steps of:
S1: collecting Raman spectra, environmental parameters, bacterial community a-diversity, and relative abundances of key bacterial taxa of a bacterial culture sample at different enrichment stages;
analyzing the Raman spectra using a multivariate curve resolution-alternating least squares (MCR-ALS) algorithm to acquire metabolite data; and
storing the metabolite data, the environmental parameters, the bacterial community a-diversity, and the relative abundances of key bacterial taxa jointly as an original dataset;
S2: performing importance calculation and data screening on the original dataset using a CART decision tree algorithm to acquire a screened dataset;
S3: optimizing and training a random forest prediction model for prediction using the screened dataset to acquire a trained random forest prediction model; and
S4: acquiring metabolite data and environmental parameters of a bacterial culture sample to be predicted, inputting the metabolite data and environmental parameters of the bacterial culture sample to be predicted jointly into the trained random forest prediction model, and outputting, by the trained random forest prediction model, prediction results of bacterial community α-diversity and relative abundances of key bacterial taxa.

In the step S1, enrichment stages of bacteria include: an adaptation stage, a growth stage, a reproduction stage, and a stabilization stage.

In the step S1, the environmental parameters include: water quality physicochemical parameters, a nutrient salt content, and OD600 bacterial concentration parameters;
the bacterial community α-diversity includes: a Shannon index, a Chao1 index, an Ace index, and a Simpson index;
the relative abundances of key bacterial taxa include: relative abundances of Anaerobic Methanotrophic Archaea (ANME), sulfate-reducing bacteria (SRB), and methanogens; and
the metabolite data include: contents of formic acid, acetic acid, methanol, formaldehyde, and sulfate.

The water quality physicochemical parameters include: contents of dissolved methane and carbon dioxide, temperature, salinity, pH, and a dissolved oxygen content; and
the nutrient salt content includes: contents of nitrate, dissolved organic matter (DOM), and total organic carbon (TOC).

In the step S1, the Raman spectra are collected using a confocal Raman microspectroscopic probe;
the water quality physicochemical parameters are collected using a multi-parameter water quality probe;
the nutrient salt content and the OD600 bacterial concentration parameters are collected using an ultraviolet spectrophotometric probe; and
DNA extraction, PCR amplification, library construction, and sequencing are sequentially performed on the bacterial culture sample at different enrichment stages to acquire DNA sequencing data; and the bacterial community α-diversity and the relative abundances of key bacterial taxa are acquired according to the DNA sequencing data.

In the step S1, the method further includes: evaluating analysis accuracy of the MCR-ALS algorithm using an MCR-BANDS algorithm.

The step S2 includes:
constructing a decision tree using the original dataset, and calculating a Gini coefficient of each decision node in the decision tree using the CART decision tree algorithm;
calculating a variable importance measure (VIM) of each data in the original dataset according to the Gini coefficient; and
performing the data screening according to the VIM, and eliminating data with a VIM lower than a preset threshold to acquire the screened dataset.

The step S3 includes:
dividing the screened dataset into the training set and the testing set at a ratio of 9:1 using a ten-fold cross-validation method;
training the random forest prediction model using the training set to acquire a random forest prediction model after training; and
evaluating performance parameters of the random forest prediction model after training using the testing set, and optimizing the random forest prediction model after training iteratively until the performance parameters meet preset conditions to acquire the trained random forest prediction model.
the performance parameters include: a coefficient of determination, a root mean square error, and a mean absolute error; and
when the coefficient of determination is larger, and the root mean square error and the mean absolute error are smaller, then performance of the random forest prediction model after training is better.

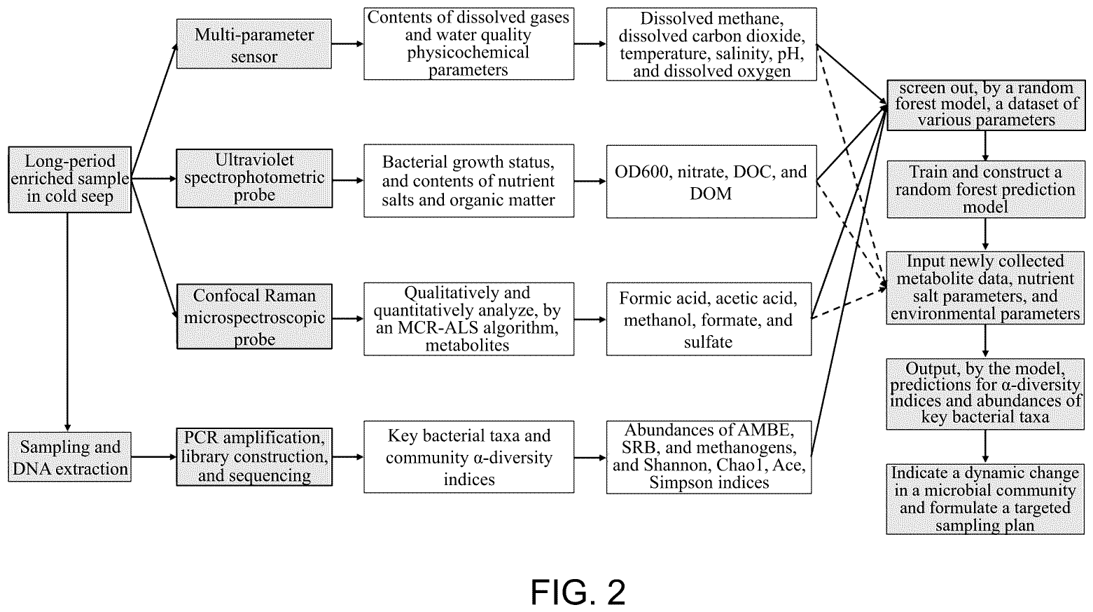

In a specific implementation process, as shown in FIG. 2, first, a long-period enriched sample in a cold seep is regularly monitored using a confocal Raman microspectroscopic probe, a multi-parameter water quality probe, and an ultraviolet spectrophotometric probe to collect original data.

In this embodiment, Raman spectroscopy is set at a wavelength of 532 nm to excite a Raman signal, with a laser intensity of 10 mW, a scanning wavenumber of 150-4200 cm⁻¹, and a collection time of 10 s. Raman spectral data collected are preprocessed by removing cosmic rays, denoising, baseline correction, and normalization;

the multi-parameter water quality probe is used to monitor and collect contents of dissolved gases (methane and carbon dioxide), temperature, salinity, pH, and a dissolved oxygen content in water; and

the ultraviolet spectrophotometric probe is used to monitor and collect a nutrient salt content and OD600 parameters of a culture sample. The nutrient salt parameters include a nitrate content and an organic carbon content. The OD600 parameter is an indicator of a bacterial growth concentration in the culture sample. A spectral range is 200-1000 nm, with a step size of 1 nm. An ultraviolet spectrum mainly collects ultraviolet spectral absorbances of nitrate (220 nm, 275 nm), DOM (355 nm, 700-800 nm, 254 nm, 250 nm, 365 nm, 440 nm, 275-295 nm), and TOC (255 nm, 265 nm, and 275 nm).

A principle of ultraviolet spectrophotometry for analyzing nitrate, DOM, and TOC is based on a dual-wavelength method for nitrate determination (220 nm and 275 nm), an ultraviolet absorption coefficient method for DOM type determination (chromophoric dissolved organic matter (CDOM), relative molecular weight of CDOM, ratio of contents of humic substances and fulvic acid in DOM, aromatic DOM), and an ultraviolet absorption method for TOC determination (255 nm, 265 nm, and 275 nm).

Calculation of an ultraviolet absorption coefficient for DOM monitoring is as follows:

$$a ( λ ) = ln 10 A ( λ ) / 1$$

where a(λ) is an absorption coefficient at a wavelength λ, with a unit of m⁻¹; A(λ) is absorbance at the wavelength λ, and a mean absorbance between 700-800 nm is subtracted to remove errors such as instrumental background noise; and 1 is a cuvette optical path length, with a unit of m.

In this embodiment, the long-period enriched sample in the cold seep is also regularly collected. Bacterial DNA from the culture sample is extracted, and PCR amplification, library construction, and sequencing of 16S rDNA sequences are performed using universal primers. Bacterial community α-diversity (Shannon index, Chaol index, Ace index, Simpson index) and relative abundances of key bacterial taxa (methane-oxidizing bacteria, sulfate-reducing bacteria, methanogens, etc.) are calculated from sequencing results to indicate a dynamic change of a bacterial community in the enriched sample.

By collecting the above Raman spectra, environmental parameters, bacterial community diversity, and abundance data of key bacterial taxa, original collected data are formed.

Based on the original collected Raman spectral data, types and concentrations of characteristic chemical components in the sample are analyzed using an MCR-ALS algorithm. Analysis of metabolites such as sulfate, formic acid, methanol, and acetic acid by Raman spectroscopy is based on pre-experiments under high-pressure conditions to fit concentration standard curves.

The MCR-ALS algorithm can decompose unknown complex spectral data of a mixed sample into pure components and concentrations through a bilinear model. Multiple grayscale thresholds are calculated from the Raman spectral data based on grayscale features using a multi-threshold segmentation method for multi-target extraction, thereby dividing the Raman spectral data into multiple parts for input. The multi-threshold segmentation method is characterized by fast calculation and high efficiency. Iterative optimization is performed using an alternating least squares (ALS) method until convergence, and robustness of models is evaluated based on an R² value (>99%) and an LOF value (<10%) of the algorithm to screen out a model with high robustness.

An MCR-ALS algorithm model is evaluated using an MCR-BANDS algorithm. A signal component contribution function (ΔSCCF value) of the MCR-BANDS algorithm can represent a degree of rotational blur. When the ΔSCCF value is closer to 0, accuracy of a model is higher, so as to evaluate the accuracy of the MCR-ALS algorithm model. Finally, based on output values of the MCR-ALS algorithm, metabolite composition and concentration data of the sample at different enrichment stages are obtained, forming a metabolite difference dataset for the sample at different enrichment stages.

Based on the metabolite data output by the MCR-ALS model, combined with the original data, an original dataset is formed including characteristic metabolite components and concentrations, environmental parameters, bacterial community diversity, and abundance data of key bacterial taxa.

A prediction model is constructed using a random forest algorithm to output results of bacterial community α-diversity (Shannon index, Chaol index, Ace index, Simpson index) and relative abundances of key functional bacterial taxa (methane-oxidizing bacteria, sulfate-reducing bacteria, methanogens, etc.) of the enriched sample.

A CART decision tree algorithm of a random forest model is used to divide the dataset according to the principle of minimum Gini index by calculating a Gini index. At the same time, a variable importance measure (VIM) of each environmental parameter is calculated based on the Gini index. Relative importance of each environmental parameter in an input dataset for an output value is compared, and environmental parameters with high importance are screened out and integrated to form a screened dataset.

The key CART decision tree algorithm calculates a Gini coefficient based on a decision node and a divided feature parameter through the following process:
a Gini index of a decision tree node for a dataset S is calculated as follows:

$$Gini ( S ) = 1 - {\sum }_{k = 1}^{k} {p}_{k}^{2}$$

where k refers to a category of the decision tree, pₖ is a probability of sampling a category k, and 1-pₖ is a probability of not sampling the category k; and
when dataset S is divided into subsets 1 and 2, a Gini coefficient for a single split is calculated based on a Gini index of a node as follows:

$${Gini}_{split} ( S ) = \frac{\vert S 1 \vert}{\vert s \vert} Gini ( S 1 ) + \frac{\vert S 1 \vert}{\vert s \vert} Gini ( S 2 )$$

Based on Giniₛₚₗᵢₜ(S), a CART decision tree in random forest is split following the principle of minimum Gini index, the splitting process is repeated until the following termination conditions are met: the number of samples in a current dataset is less than a certain given value; all samples in the current dataset belong to a same category; attribute variables of the current dataset are 0; and the depth of the decision tree exceeds a certain given value.

For each environmental parameter in the dataset, a variable importance measure (VIM) of an environmental parameter is calculated using Gini indices of each node and split:

$${VIM}_{n}^{( Gini ) ( i )} = {\sum }_{s ∈ S} {VIM}_{ns}^{( Gini ) ( i )} = {\sum }_{s ∈ S} Gini ( S ) - {Gini}_{split} ( S )$$

VIMₙ^{(Gini)(i)} refers to importance of a characteristic variable Xₙ in an i-th decision tree, where S is a set of nodes where the characteristic variable appears in the i-th decision tree, and s represents each node in the set S.

In random forest operations, variable importance measures of features across decision trees are normalized to compare relative importance of each parameter to an overall decision:

$${VIM}_{n}^{( Gini )} = \frac{{\sum }_{i = 1}^{I} {VIM}_{n}^{( Gini ) ( i )}}{{\sum }_{n = 1}^{N} ( {\sum }_{i = 1}^{I} {VIM}_{n}^{( Gini ) ( i )} )}$$

VIMₙ^{(Gini)} refers to relative importance of the characteristic variable Xₙ among variables, and VIMₙ^{(Gini)(i)} refers to the importance of the characteristic variable Xₙ in the i-th decision tree.

Subsequently, following a ten-fold cross-validation method, the screened dataset is split into: a 90% training set for training and constructing a random forest model, and a 10% testing set for testing an output result of the model. Parameters of the random forest model (number of sub-datasets, maximum number of features, etc.) are optimized iteratively through the training set to improve prediction accuracy of the model. Based on evaluation parameters of the ten-fold cross-validation method (coefficient of determination R², root mean square error (RMSE), mean absolute error (MAE)), models are compared. A model with a larger R² value and smaller RMSE and MAE values is selected as an optimal random forest prediction model.

The evaluation parameters of the ten-fold cross-validation method are calculated as follows:

$$\begin{matrix}{R}^{2} = 1 - \frac{{\sum }_{a = 1}^{n} {( {y}_{a} - {y}_{a 1} )}^{2}}{{\sum }_{a = 1}^{n} {( {y}_{a} - {y}_{a 2} )}^{2}} \\ RMSE = \sqrt{\frac{{\sum }_{a = 1}^{n} {( {y}_{a} - {y}_{a 1} )}^{2}}{n}} \\ MAE = {\sum }_{a = 1}^{n} \vert \frac{{y}_{a} - {y}_{a 1}}{n} \vert\end{matrix}$$

where R² is the coefficient of determination, RMSE is the root mean square error, MAE is the mean absolute error, in the formula, yₐ is an a-th measured value, yₐ₁ is an a-th model predicted value, and yₐ₂ is an arithmetic mean of the a-th measured value. When the R² value is higher, the RMSE and MAE values are lower, indicating better testing performance of the model.

In a long-period enrichment process in the cold seep, the confocal Raman microspectroscopic probe, multi-parameter water quality probe, and ultraviolet spectrophotometric probe are used periodically to collect parameters such as Raman spectra (metabolite composition and concentrations resolved based on the MCR-ALS algorithm), OD600, nutrient salt contents (nitrate, sulfate, DOC), DOM types (aromaticity, hydrophobicity), contents of dissolved gases (methane and carbon dioxide), temperatures, salinity levels, pH, and dissolved oxygen contents from different enriched samples to form online monitoring data.

The online-obtained data are input into the optimal random forest prediction model, which outputs predictions for the bacterial community α-diversity indices (Shannon index, Chaol index, Ace index, Simpson index) and the relative abundances of key functional bacterial taxa (methane-oxidizing bacteria, sulfate-reducing bacteria, methanogens, etc.). Compared with traditional methods involving sampling, DNA extraction, library construction, and sequencing, this method offers advantages in accuracy, efficiency, and reduced sample consumption. Prediction data output by the random forest model can be used to indicate the dynamic change of the bacterial community and growth and reproduction status of key bacteria in the enrichment stage sample, and then identify an inflection point of a long-period enrichment stage in the cold seep and formulate a targeted sampling strategy to improve sampling efficiency of the enriched sample.

### Embodiment 3

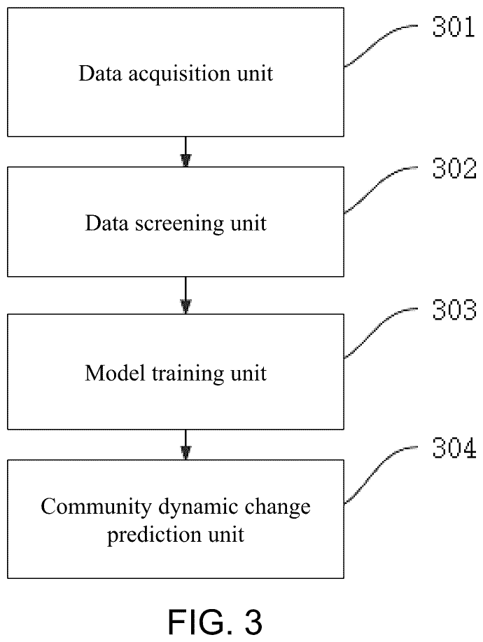

As shown in FIG. 3, this example provides a system for predicting a community dynamic change of a long-period enriched bacterial community in a cold seep, applying the method for predicting a community dynamic change of a long-period enriched bacterial community in a cold seep described in Example 1 or 2, and including:
a data acquisition unit 301: configured to collect Raman spectra, environmental parameters, bacterial community α-diversity, and relative abundances of key bacterial taxa of a bacterial culture sample at different enrichment stages;
analyze the Raman spectra using an MCR-ALS algorithm to acquire metabolite data; and
store the metabolite data, the environmental parameters, the bacterial community a-diversity, and the relative abundances of key bacterial taxa jointly as an original dataset;
a data screening unit 302: configured to perform importance calculation and data screening on the original dataset using a CART decision tree algorithm to acquire a screened dataset;
a model training unit 303: configured to optimize and train a random forest prediction model for prediction using the screened dataset to acquire a trained random forest prediction model; and
a community dynamic change prediction unit 304: configured to acquire metabolite data and environmental parameters of a bacterial culture sample to be predicted, input the metabolite data and environmental parameters of the bacterial culture sample jointly into the trained random forest prediction model, and output, by the trained random forest prediction model, prediction results of bacterial community α-diversity and relative abundances of key bacterial taxa.

In a specific implementation process, first, a data acquisition unit 301 collects Raman spectra, environmental parameters, bacterial community α-diversity, and relative abundances of key bacterial taxa of a long-period bacterial culture sample at different enrichment stages; analyzes the Raman spectra using an MCR-ALS algorithm to acquire metabolite data; and stores the metabolite data, the environmental parameters, the bacterial community α-diversity, and the relative abundances of key bacterial taxa jointly as an original dataset.

Next, a data screening unit 302 performs importance calculation and data screening on the original dataset using a CART decision tree algorithm to acquire a screened dataset.

Then, a model training unit 303 optimizes and trains a random forest prediction model using the screened dataset to acquire a trained random forest prediction model.

Finally, a community dynamic change prediction unit 304 acquires metabolite data and environmental parameters of a bacterial culture sample to be predicted, inputs them jointly into the trained random forest prediction model, and outputs, by the trained random forest prediction model, prediction results of bacterial community α-diversity and relative abundances of key bacterial taxa.

In this system, a dataset is formed through routine sampling and collection of a complete bacterial community succession process (adaptation, growth, reproduction, and stabilization) in an early stage of enrichment culture, and an accurate prediction model is constructed by using a machine learning algorithm, thereby improving sampling efficiency of a long-period enrichment culture operation for a cold seep bacterial community.

Identical or similar reference numerals correspond to identical or similar components.

Terms describing positional relationships in the drawings are for exemplary illustration only and shall not be construed as limitations to this patent.

Obviously, the above embodiments of the present disclosure are merely examples provided for clear explanation, rather than limitations to the implementations of the present disclosure. For those of ordinary skill in the art, various other forms of changes or alterations can be made based on the above description. It is neither necessary nor possible to enumerate all implementations here. Any modifications, equivalent substitutions, improvements, etc. made within the spirit and principles of the present disclosure shall be included in the protection scope of the claims of the present disclosure.

What is claimed is:

1. A method for predicting a community dynamic change of a long-period enriched bacterial community in a cold seep, comprising steps of:
S1: collecting Raman spectra, environmental parameters, bacterial community α-diversity, and relative abundances of key bacterial taxa of a bacterial culture sample at different enrichment stages, wherein the relative abundances of the key bacterial taxa comprise: relative abundances of Anaerobic Methanotrophic Archaea (ANME), sulfate-reducing bacteria (SRB), and methanogens;
analyzing the Raman spectra using an MCR-ALS algorithm to acquire metabolite data; and
storing the metabolite data, the environmental parameters, the bacterial community a-diversity, and the relative abundances of the key bacterial taxa jointly as an original dataset;
S2: performing importance calculation and data screening on the original dataset using a CART decision tree algorithm to acquire a screened dataset;
S3: optimizing and training a random forest prediction model for prediction using the screened dataset to acquire a trained random forest prediction model; and
S4: acquiring metabolite data and environmental parameters of a bacterial culture sample to be predicted, inputting the metabolite data and the environmental parameters of the bacterial culture sample to be predicted jointly into the trained random forest prediction model, and outputting, by the trained random forest prediction model, prediction results of the bacterial community α-diversity and the relative abundances of the key bacterial taxa.

2. The method for predicting the community dynamic change of the long-period enriched bacterial community in the cold seep according to claim 1, wherein in the step S1, enrichment stages of bacteria comprise: an adaptation stage, a growth stage, a reproduction stage, and a stabilization stage.

3. The method for predicting the community dynamic change of the long-period enriched bacterial community in the cold seep according to claim 1, wherein in the step S1, the environmental parameters comprise: water quality physicochemical parameters, a nutrient salt content, and OD600 bacterial concentration parameters;
the bacterial community α-diversity comprises: a Shannon index, a Chaol index, an Ace index, and a Simpson index; and
the metabolite data comprise: contents of formic acid, acetic acid, methanol, formaldehyde, and sulfate.

4. The method for predicting the community dynamic change of the long-period enriched bacterial community in the cold seep according to claim 3, wherein the water quality physicochemical parameters comprise: contents of dissolved methane and carbon dioxide, temperature, salinity, pH, and a dissolved oxygen content; and
the nutrient salt content comprises: contents of nitrate, dissolved organic matter (DOM), and total organic carbon (TOC).

5. The method for predicting the community dynamic change of the long-period enriched bacterial community in the cold seep according to claim 4, wherein the Raman spectra are collected using a confocal Raman microspectroscopic probe;
the water quality physicochemical parameters are collected using a multi-parameter water quality probe;
the nutrient salt content and the OD600 bacterial concentration parameters are collected using an ultraviolet spectrophotometric probe; and
DNA extraction, PCR amplification, library construction, and sequencing are sequentially performed on the bacterial culture sample at different enrichment stages to acquire DNA sequencing data; and the bacterial community α-diversity and the relative abundances of the key bacterial taxa are acquired according to the DNA sequencing data.

6. The method for predicting the community dynamic change of the long-period enriched bacterial community in the cold seep according to claim 1, wherein in the step S1, the method further comprises: evaluating analysis accuracy of the MCR-ALS algorithm using an MCR-BANDS algorithm.

7. The method for predicting the community dynamic change of the long-period enriched bacterial community in the cold seep according to claim 1, wherein the step S2 comprises:
constructing a decision tree using the original dataset, and calculating a Gini coefficient of each decision node in the decision tree using the CART decision tree algorithm;
calculating a variable importance measure (VIM) of each data in the original dataset according to the Gini coefficient; and
performing the data screening according to the VIM, and eliminating data with a VIM lower than a preset threshold to acquire the screened dataset.

8. The method for predicting the community dynamic change of the long-period enriched bacterial community in the cold seep according to claim 1, wherein the step S3 comprises:
dividing the screened dataset into a training set and a testing set;
training the random forest prediction model using the training set to acquire a random forest prediction model after training; and
evaluating performance parameters of the random forest prediction model after training using the testing set, and optimizing the random forest prediction model after training iteratively until the performance parameters meet preset conditions to acquire the trained random forest prediction model.

9. The method for predicting the community dynamic change of the long-period enriched bacterial community in the cold seep according to claim 8, wherein the screened dataset is divided into the training set and the testing set at a ratio of 9:1 using a ten-fold cross-validation method;
the performance parameters comprise: a coefficient of determination, a root mean square error, and a mean absolute error; and
when the coefficient of determination is larger, and the root mean square error and the mean absolute error are smaller, then performance of the random forest prediction model after training is better.

10. A system for predicting a community dynamic change of a long-period enriched bacterial community in a cold seep, applying a method for predicting the community dynamic change of the long-period enriched bacterial community in the cold seep according to claim 1, and comprising:
a data acquisition unit: configured to collect Raman spectra, environmental parameters, bacterial community α-diversity, and relative abundances of key bacterial taxa of a bacterial culture sample at different enrichment stages;
analyze the Raman spectra using an MCR-ALS algorithm to acquire metabolite data; and
store the metabolite data, the environmental parameters, the bacterial community α-diversity, and the relative abundances of the key bacterial taxa jointly as an original dataset;
a data screening unit: configured to perform importance calculation and data screening on the original dataset using a CART decision tree algorithm to acquire a screened dataset;
a model training unit: configured to optimize and train a random forest prediction model for prediction using the screened dataset to acquire a trained random forest prediction model; and
a community dynamic change prediction unit: configured to acquire metabolite data and environmental parameters of a bacterial culture sample to be predicted, input the metabolite data and the environmental parameters of the bacterial culture sample to be predicted jointly into the trained random forest prediction model, and output, by the trained random forest prediction model, prediction results of the bacterial community α-diversity and the relative abundances of the key bacterial taxa.

11. The method for predicting the community dynamic change of the long-period enriched bacterial community in the cold seep according to claim 5, wherein in the step S1, the method further comprises: evaluating analysis accuracy of the MCR-ALS algorithm using an MCR-BANDS algorithm.

12. A system for predicting a community dynamic change of a long-period enriched bacterial community in a cold seep, applying a method for predicting the community dynamic change of the long-period enriched bacterial community in the cold seep according to claim 2, and comprising:
a data acquisition unit: configured to collect Raman spectra, environmental parameters, bacterial community α-diversity, and relative abundances of key bacterial taxa of a bacterial culture sample at different enrichment stages;
analyze the Raman spectra using an MCR-ALS algorithm to acquire metabolite data; and
store the metabolite data, the environmental parameters, the bacterial community α-diversity, and the relative abundances of the key bacterial taxa jointly as an original dataset;
a data screening unit: configured to perform importance calculation and data screening on the original dataset using a CART decision tree algorithm to acquire a screened dataset;
a model training unit: configured to optimize and train a random forest prediction model for prediction using the screened dataset to acquire a trained random forest prediction model; and
a community dynamic change prediction unit: configured to acquire metabolite data and environmental parameters of a bacterial culture sample to be predicted, input the metabolite data and the environmental parameters of the bacterial culture sample to be predicted jointly into the trained random forest prediction model, and output, by the trained random forest prediction model, prediction results of the bacterial community α-diversity and the relative abundances of the key bacterial taxa.

13. A system for predicting a community dynamic change of a long-period enriched bacterial community in a cold seep, applying a method for predicting the community dynamic change of the long-period enriched bacterial community in the cold seep according to claim 3, and comprising:
a data acquisition unit: configured to collect Raman spectra, environmental parameters, bacterial community α-diversity, and relative abundances of key bacterial taxa of a bacterial culture sample at different enrichment stages;
analyze the Raman spectra using an MCR-ALS algorithm to acquire metabolite data; and
store the metabolite data, the environmental parameters, the bacterial community α-diversity, and the relative abundances of the key bacterial taxa jointly as an original dataset;
a data screening unit: configured to perform importance calculation and data screening on the original dataset using a CART decision tree algorithm to acquire a screened dataset;
a model training unit: configured to optimize and train a random forest prediction model for prediction using the screened dataset to acquire a trained random forest prediction model; and
a community dynamic change prediction unit: configured to acquire metabolite data and environmental parameters of a bacterial culture sample to be predicted, input the metabolite data and the environmental parameters of the bacterial culture sample to be predicted jointly into the trained random forest prediction model, and output, by the trained random forest prediction model, prediction results of the bacterial community α-diversity and the relative abundances of the key bacterial taxa.

14. A system for predicting a community dynamic change of a long-period enriched bacterial community in a cold seep, applying a method for predicting the community dynamic change of the long-period enriched bacterial community in the cold seep according to claim 4, and comprising:
a data acquisition unit: configured to collect Raman spectra, environmental parameters, bacterial community α-diversity, and relative abundances of key bacterial taxa of a bacterial culture sample at different enrichment stages;
analyze the Raman spectra using an MCR-ALS algorithm to acquire metabolite data; and
store the metabolite data, the environmental parameters, the bacterial community α-diversity, and the relative abundances of the key bacterial taxa jointly as an original dataset;
a data screening unit: configured to perform importance calculation and data screening on the original dataset using a CART decision tree algorithm to acquire a screened dataset;
a model training unit: configured to optimize and train a random forest prediction model for prediction using the screened dataset to acquire a trained random forest prediction model; and
a community dynamic change prediction unit: configured to acquire metabolite data and environmental parameters of a bacterial culture sample to be predicted, input the metabolite data and the environmental parameters of the bacterial culture sample to be predicted jointly into the trained random forest prediction model, and output, by the trained random forest prediction model, prediction results of the bacterial community α-diversity and the relative abundances of the key bacterial taxa.

15. A system for predicting a community dynamic change of a long-period enriched bacterial community in a cold seep, applying a method for predicting the community dynamic change of the long-period enriched bacterial community in the cold seep according to claim 5, and comprising:
a data acquisition unit: configured to collect Raman spectra, environmental parameters, bacterial community α-diversity, and relative abundances of key bacterial taxa of a bacterial culture sample at different enrichment stages;
analyze the Raman spectra using an MCR-ALS algorithm to acquire metabolite data; and
store the metabolite data, the environmental parameters, the bacterial community α-diversity, and the relative abundances of the key bacterial taxa jointly as an original dataset;
a data screening unit: configured to perform importance calculation and data screening on the original dataset using a CART decision tree algorithm to acquire a screened dataset;
a model training unit: configured to optimize and train a random forest prediction model for prediction using the screened dataset to acquire a trained random forest prediction model; and
a community dynamic change prediction unit: configured to acquire metabolite data and environmental parameters of a bacterial culture sample to be predicted, input the metabolite data and the environmental parameters of the bacterial culture sample to be predicted jointly into the trained random forest prediction model, and output, by the trained random forest prediction model, prediction results of the bacterial community α-diversity and the relative abundances of the key bacterial taxa.

16. A system for predicting a community dynamic change of a long-period enriched bacterial community in a cold seep, applying a method for predicting the community dynamic change of the long-period enriched bacterial community in the cold seep according to claim 6, and comprising:
a data acquisition unit: configured to collect Raman spectra, environmental parameters, bacterial community α-diversity, and relative abundances of key bacterial taxa of a bacterial culture sample at different enrichment stages;
analyze the Raman spectra using an MCR-ALS algorithm to acquire metabolite data; and
store the metabolite data, the environmental parameters, the bacterial community α-diversity, and the relative abundances of the key bacterial taxa jointly as an original dataset;
a data screening unit: configured to perform importance calculation and data screening on the original dataset using a CART decision tree algorithm to acquire a screened dataset;
a model training unit: configured to optimize and train a random forest prediction model for prediction using the screened dataset to acquire a trained random forest prediction model; and
a community dynamic change prediction unit: configured to acquire metabolite data and environmental parameters of a bacterial culture sample to be predicted, input the metabolite data and the environmental parameters of the bacterial culture sample to be predicted jointly into the trained random forest prediction model, and output, by the trained random forest prediction model, prediction results of the bacterial community α-diversity and the relative abundances of the key bacterial taxa.

17. A system for predicting a community dynamic change of a long-period enriched bacterial community in a cold seep, applying a method for predicting the community dynamic change of the long-period enriched bacterial community in the cold seep according to claim 7, and comprising:
a data acquisition unit: configured to collect Raman spectra, environmental parameters, bacterial community α-diversity, and relative abundances of key bacterial taxa of a bacterial culture sample at different enrichment stages;
analyze the Raman spectra using an MCR-ALS algorithm to acquire metabolite data; and
store the metabolite data, the environmental parameters, the bacterial community α-diversity, and the relative abundances of the key bacterial taxa jointly as an original dataset;
a data screening unit: configured to perform importance calculation and data screening on the original dataset using a CART decision tree algorithm to acquire a screened dataset;
a model training unit: configured to optimize and train a random forest prediction model for prediction using the screened dataset to acquire a trained random forest prediction model; and
a community dynamic change prediction unit: configured to acquire metabolite data and environmental parameters of a bacterial culture sample to be predicted, input the metabolite data and the environmental parameters of the bacterial culture sample to be predicted jointly into the trained random forest prediction model, and output, by the trained random forest prediction model, prediction results of the bacterial community α-diversity and the relative abundances of the key bacterial taxa.

18. A system for predicting a community dynamic change of a long-period enriched bacterial community in a cold seep, applying a method for predicting the community dynamic change of the long-period enriched bacterial community in the cold seep according to claim 8, and comprising:
a data acquisition unit: configured to collect Raman spectra, environmental parameters, bacterial community α-diversity, and relative abundances of key bacterial taxa of a bacterial culture sample at different enrichment stages;
analyze the Raman spectra using an MCR-ALS algorithm to acquire metabolite data; and
store the metabolite data, the environmental parameters, the bacterial community α-diversity, and the relative abundances of the key bacterial taxa jointly as an original dataset;
a data screening unit: configured to perform importance calculation and data screening on the original dataset using a CART decision tree algorithm to acquire a screened dataset;
a model training unit: configured to optimize and train a random forest prediction model for prediction using the screened dataset to acquire a trained random forest prediction model; and
a community dynamic change prediction unit: configured to acquire metabolite data and environmental parameters of a bacterial culture sample to be predicted, input the metabolite data and the environmental parameters of the bacterial culture sample to be predicted jointly into the trained random forest prediction model, and output, by the trained random forest prediction model, prediction results of the bacterial community α-diversity and the relative abundances of the key bacterial taxa.

19. A system for predicting a community dynamic change of a long-period enriched bacterial community in a cold seep, applying a method for predicting the community dynamic change of the long-period enriched bacterial community in the cold seep according to claim 9, and comprising:
a data acquisition unit: configured to collect Raman spectra, environmental parameters, bacterial community α-diversity, and relative abundances of key bacterial taxa of a bacterial culture sample at different enrichment stages;
analyze the Raman spectra using an MCR-ALS algorithm to acquire metabolite data; and
store the metabolite data, the environmental parameters, the bacterial community α-diversity, and the relative abundances of the key bacterial taxa jointly as an original dataset;
a data screening unit: configured to perform importance calculation and data screening on the original dataset using a CART decision tree algorithm to acquire a screened dataset;
a model training unit: configured to optimize and train a random forest prediction model for prediction using the screened dataset to acquire a trained random forest prediction model; and
a community dynamic change prediction unit: configured to acquire metabolite data and environmental parameters of a bacterial culture sample to be predicted, input the metabolite data and the environmental parameters of the bacterial culture sample to be predicted jointly into the trained random forest prediction model, and output, by the trained random forest prediction model, prediction results of the bacterial community α-diversity and the relative abundances of the key bacterial taxa.
